SEQUENCE LISTING (1 sequence)

SEQ ID NO: 1 - DNA, 22 bp, Unknown
  NF- kappaB consensus binding sequence
agttgaggggactttcccaggc

(19) United States
(12) Patent Application Publication
(10) Pub. No.: US 2008/0138280 A1
(43) Pub. Date: Jun. 12, 2008

(54) Composition and Methods For Inhibiting Cell Survival

(75) Inventor: Joseph A. Bauer, Akron, OH (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: The Cleveland Clinic Foundation, Cleveland, OH (US)

(21) Appl. No.: 10/546,094

(22) PCT Filed: Feb. 19, 2004
(86) PCT No.: PCT/US04/04988

§ 371 Date: Jun. 30, 2006

Related U.S. Application Data
(60) Provisional application No. 60/448,501, filed on Feb. 20, 2003.

(51) Int. Cl. A61K 31/7056; A61K 39/395; A61K 33/24; A61K 38/21; A61K 51/04; A61P 35/00

(52) U.S. Cl. 424/1.77; 514/52; 424/141.1; 424/649; 424/85.6

(57) ABSTRACT

The present invention is directed to compositions and methods of making and using compositions that are useful for treating cells or conditions caused or exacerbated by cell survival mechanisms of the body, particularly conditions exacerbated by cell survival mechanisms involving NF-KB. The compositions and methods of the present invention comprise a sensitizing agent or a chemopotentiating agent. In this regard, cobalamin drug conjugates and NO donors act as chemopotentiating agents. Nitrosylcobalamin is particularly useful as a sensitizing or chemopotentiating agent, and methods utilizing nitrosylcobalamin prior to, simultaneous with and subsequent to radiation or chemotherapy are described, as are compositions which first release a nitrosylcobalamin compound or biologically active analog thereof, and then release the chemotherapeutic agent. Nitrosylcobalamin itself is a chemotherapeutic, and when administered in conjunction with other anti-cancer agents or techniques, a synergistic effect is seen.

### CROSS REFERENCE TO RELATED APPLICATIONS

The present application claims priority to U.S. Provisional Application No. 60/448,501 as filed on Feb. 20, 2003. The disclosure of the U.S. Provisional Application No. 60/448,501 is incorporated herein by reference in its entirety.

### BACKGROUND OF TE INVENTION

Metallocorrinoids are corrin rings with a metal-atom center, such as Co, Fe, Ni, or Mn. A corrin ring is four reduced pyrrole rings linked together. A subclass of naturally occurring metallocorrinoids is known as cobalamin, that is, a cobalt-centered corrin ring. Naturally occurring vitamin B₁₂, for example, is a cobalamin. Vitamin B₁₂ compounds are known to have many biological functions. They are required by the enzyme methionine synthase, for example, which is involved in the production of DNA. It is believed that vitamin B₁₂ enhances the effects of other vitamins and nutrients in tissue repair.

Cobalamin (Vitamin B₁₂ or “Cbl”), an essential micronutrient, is important in maintaining differentiation, proliferation and metabolic status of cells. Circulating Cbl are known to bind to plasma transcobalamin II (TC II). A 43 kDa non-glycosylated protein may be taken up by receptor mediated endocytosis in all cells, via a specific receptor, TC II-receptor (TC II-R). Following endocytosis of TC II-Cbl, TC II is degraded in the lysosomes and the Cbl liberated is converted to its coenzyme forms, methyl-Cbl and 5′-deoxyadenosyl-Cbl. Methyl-Cbl is utilized for the conversion of homocysteine to methionine by the enzyme methionine synthase; 5′-deoxyadenosyl-Cbl is used for the conversion of methylmalonyl CoA to succinyl CoA, an important intermediate of the tricarboxylic acid cycle by the enzyme methylmalonyl CoA mutase. Intracellular Cbl deficiency results in multiple organ disorders that include hematological (reticulocytes), immunological (lymphocytes), gastrointestinal (absorptive epithelial) and neurological (glial) defects. Impaired DNA synthesis is associated with the onset of megaloblastosis.

The TC II(TC II-R delivery system of Cbl plays an important role in Cbl uptake in transformed cells. Cbl accumulation occurs preferentially in tumors. Autoradiography of histologic sections demonstrate an increased affinity for Cbl by some tumors in vivo. The accumulation of Cbl in tumors has recently been confirmed using radioimaging studies in rats and humans using radiolabeled Cbl analogues to detect occult tumors. Also, methionine-dependent human glial cells that are like cancer cells have an imbalance between methionine synthesis and utilization and cease to proliferate in the absence of methionine in the medium. However, when cultured in the presence of homocysteine, an immediate precursor of methionine, these cells demonstrate increased TC II-R activity and Cbl import. Certain Cbl analogues have antiproliferative activity against leukemia cells. In addition, monoclonal antibodies against TC II that block its binding to TC II-R can block the proliferation of leukemic cells. Taken together, these studies suggest that increased Cbl delivery in cancer cells may be due to increased TC II-R number or density on the cancer cell plasma membrane.

Cobalamin analogs and cobalamin drug conjugates have been shown to inhibit the growth of leukemia cells by possibly deactivating methionine synthase, thus preventing DNA synthesis. All forms of vitamin B₁₂ (adenosyl-, cyano-, hydroxo-, or methylcobalamin) are bound by the transport proteins intrinsic factor and transcobalamin II, to be biologically active. Those transport proteins involved in the uptake of vitamin B₁₂ are referred to herein as cobalamin binding proteins. Specifically, gastrointestinal absorption of vitamin B₁₂ relies upon the intrinsic factor-vitamin B₁₂ complex being bound by the intrinsic factor receptors in the terminal ileum. Likewise, intravascular transport and subsequent cellular uptake of vitamin B₁₂ throughout the body is dependent upon transcobalamin II and the cell membrane transcobalamin II receptors, respectively. After the transcobalamin II-vitamin B₁₂ complex has been internalized, the transport protein undergoes lysozymal degradation, which releases vitamin B₁₂ into the cytoplasm.

Cobalamin analogs and cobalamin drug conjugates suitable in the present invention may include radiolabeled vitamin B₁₂ analogs, which have been described in the art as useful in vivo imaging agents. For example, U.S. Pat. No. 6,096,290, which is hereby incorporated herein in its entirety by reference thereto, describes the use of radiolabelled vitamin B₁₂ analogs as in vivo tumor imaging agents.

U.S. Pat. No. 6,183,723, which is also incorporated herein by reference in its entirety, describes certain other cobalamin-drug conjugates suitable in the present invention.

U.S. Pat. No. 5,936,082, which is hereby incorporated by reference in its entirety, for example, describes the therapeutic effectiveness of vitamin B₁₂ based compounds. Nitrosylcobalamin (NO-Cbl), in particular, was evaluated in U.S. Pat. No. 5,936,082 for its chemotherapeutic effect. In human hematological and solid tumor cell lines, NO-Cbl exhibited an ID₅₀ that was 5-100 fold lower in tumor cell lines compared to benign cells (fibroblasts and endothelial cells). When oxidized from NO-Cbl, the NO free radical functions in a number of capacities. NO is involved in vasodilation, and is known to contribute to increased oxidative stress, inhibition of cellular metabolism and induction of DNA damage leading to apoptosis and/or necrosis.

U.S. patent application Ser. No. 09/864,747 and the corresponding PCT Publication WO 02/094309, both of which are incorporated herein in their entirety by reference thereto, describe composition and methods for enhancing the uptake of cobalamin and cobalamin drug conjugates.

It has been found that cobalamin drug conjugates are useful as chemopotentiating agents.

### SUMMARY OF THE INVENTION

The present invention is directed generally to the use of NO donors and cobalamin drug conjugates, such as Vitamin B₁₂ or Vitamin B₁₂ analogs, as chemopotentiating agents. Accordingly, an aspect of the present invention is a therapeutic composition comprising a chemopotentiating cobalamin drug conjugate. Another aspect of the present invention is a therapeutic composition comprising a chemopotentiating NO donor. Another aspect of the present invention is a therapeutic composition comprised of a cobalamin drug conjugate and a chemotherapeutic agent. Another aspect of the present invention is a therapeutic composition comprising a chemopotentiating NO donor and a chemotherapeutic agent. A preferred embodiment of the present invention is a therapeutic composition comprised of nitrosylcobalamin and a chemotherapeutic agent. Another preferred embodiment of the present invention is a therapeutic composition comprised of nitrosylcobalamin and Apo2L/TRAIL or a therapeutic composition comprised of nitrosylcobalamin and a cytokine.

Another embodiment of the present invention is a method of inhibiting tumor growth in vivo comprised of administering a chemopotentiating agent such as a cobalamin drug conjugate or a NO donor. Another embodiment of the present invention is a method of treating a patient with a condition comprising the steps of sensitizing the patient to chemotherapy or radiation by administering a cobalamin drug conjugate or a NO donor and subsequently administering a chemotherapeutic agent or radiation. Another embodiment of the present invention is a method of inhibiting NF-κB activation comprised of administering a cobalamin drug conjugate or a NO donor. Another embodiment of the present invention is a method of inhibiting cell survival signaling comprised of administering a cobalamin drug conjugate or a NO donor. Another embodiment of the present invention is a method of treating cancer comprised of administering a composition of nitrosylcobalamin and a chemotherapeutic agent. Another embodiment of the present invention is a method of treating cancer comprised of administering a composition of nitrosylcobalamin and a cytokine or Apo2L/TRAIL.

An embodiment of the present invention is the use of a cobalamin drug conjugate or a NO donor to sensitize cells to the anti-tumor effects of chemotherapeutic drugs, agents and procedures. One embodiment of the present invention is a therapeutic composition comprising a chemopotentiating agent.

Another embodiment of the invention is a therapeutic composition comprising a chemopotentiating agent and a chemotherapeutic agent, wherein the chemopotentiating agent may be radiolabeled vitamin B₁₂, nitrosylcobalamin, hydroxocobalamin, cyanocobalamin, nitrocobalamin, methylcobalamin, or 5-desoxyadenosylcobalamin. Suitable radiolabeled vitamin B₁₂ compounds include homologs, analogs and derivatives.

Another embodiment of the present invention is the use of a nitric oxide donor as a chemopotentiating agent. Suitable nitric oxide donors include nitrosylcobalamin, SNP, SNAP, and NOC 18. NO donors may be used in connection with a chemotherapeutic agent to inhibit tumor growth. NO donors sensitizes cells to the anti-tumor effects of chemotherapeutic agents and procedures.

Aspects and applications of the present invention will become apparent to the skilled artisan upon consideration of the detailed description of the invention and the figures accompanying the description, which follows.

### BRIEF DESCRIPTION OF THE DRAWINGS

FIG. 1 is a chart summarizing the median effect analysis on the chemopotentiating effects of NO-Cbl and various chemopotentiating agents over a number of cell lines;

FIG. 2 illustrates the effect of NO-Cbl, Apo2L/TRAIL and the combination on the growth of tumor volume in vivo;

FIG. 3 illustrates a TUNEL apoptosis assay in accordance with the present invention. A375 cells were treated with NO-Cbl, Apo2L/TRAIL, and the combination. NO-Cbl and Apo2L/TRAIL were minimally effective as single agents but demonstrated greater apoptosis when administered concomitantly; however A375 cells pre-treated with NO-Cbl followed by Apo2L/TRAIL demonstrated the greatest amount of apoptosis;

FIG. 4 is a Western blot analysis of mediators of apoptosis. a, A375 cells were pre-treated with NO-Cbl, followed by Apo2L/TRAIL which resulted in cleavage of caspase-3, caspase-8, and PARP. b, Sequential NO-Cbl and Apo2L/TRAIL treatment caused cleavage of XIAP, an inhibitor of apoptosis;

FIG. 5 illustrates an Electrophoretic Mobility Shift Assay (EMSA): NF-κB DNA binding activity. a, Pre-treatment of A375 cells with NO-Cbl inhibited the NF-κB DNA binding activity induced by Apo2L/TRAIL and TNF-α. b, NO donors, NOC-18 and SNAP also reduced Apo2L/TRAIL-induced NF-κB DNA binding. c, NF-κB-luc transfected A375 cells were pre-treated with NO-Cbl followed by Apo2L/TRAIL or TNF-α. Renilla luciferase was co-transfected to normalize samples for transfection efficiency. Cell lysates were analyzed for NF-κB-luc reporter activity. NO-Cbl pre-treatment inhibited Apo2L/TRAIL and TNF-α induced activation of the NF-κB luc reporter;

FIG. 6 is a Western blot analysis of IκB levels and IκBα phosphorylation. IκBα and phospho-IκBα protein levels were determined in A375 whole cell lysates. a, Cells pre-treated with NO-Cbl exhibited decreased levels of phosphorylated IκBα following Apo2L/TRAIL or TNF-α stimulation. b, NO-Cbl, NOC-18, and SNAP pre-treatment all inhibited Apo2L/TRAIL-induced IκBα phosphorylation.

FIG. 7 illustrates that NO-Cbl sensitizes cancer cells to irradiation.

FIG. 8 illustrates that NO-Cbl inhibited IκB kinase (IKK) activity which was stimulated with Apo2L/TRAIL or TNF-α, using recombinant GST-IκBα-(1-54) and [γ³²P]ATP as substrates.

FIG. 9 is an Electrophoretic Mobility Shift Assay of NF-κB DNA binding activity, wherein NO-Cbl inhibited the NF-κB DNA binding activity induced by stimulation with CPT or VP16.

FIG. 10 is an Electrophoretic Mobility Shift Assay of NF-κB DNA binding activity, wherein NO-Cbl inhibited the NF-κB DNA binding activity induced by stimulation with doxorubicin.

### DETAILED DESCRIPTION OF THE INVENTION

For simplicity and illustrative purposes, the principles of the invention are described by referring mainly to an embodiment thereof. In addition, in the following description, numerous specific details are set forth in order to provide a thorough understanding of the invention. It will be apparent however, to one of ordinary skill in the art, that the invention may be practiced without limitation to these specific details. In other instances, well known methods and structures have not been described in detail so as not to unnecessarily obscure the invention.

It must also be noted that as used herein and in the appended claims, the singular forms “a”, “an”, and “the” include plural reference unless the context clearly dictates otherwise. Unless defined otherwise, all technical and scientific terms used herein have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

The present invention is directed to compositions and methods of making and using compositions that are useful for treating cells or conditions caused or exacerbated by cell survival mechanisms of the body, particularly conditions exacerbated by cell survival mechanisms, such as mechanisms involving NF-κB. The compositions and methods of the present invention are directed to the use of a sensitizing agent or a chemopotentiating agent. For example, nitrosylcobalamin may be used as a sensitizing or chemopotentiating agent, and methods utilizing nitrosylcobalamin(s) prior to, simultaneous with and subsequent to radiation or chemotherapy are described, as are compositions which first release a nitrosylcobalamin compound or biologically active analog thereof, and then release the chemotherapeutic agent. Nitrosylcobalamin itself is a chemotherapeutic, and when administered in conjunction with other anti-cancer agents or techniques, a synergistic effect is seen.

The term “chemopotentiating agent” refers to an agent that acts to increase the sensitivity of an organism, tissue, or cell to a chemical compound, or treatment namely “chemotherapeutic agents” or “chemo drugs” or radiation treatment.

Chemopotentiating agents suitable in embodiments of the present invention include cobalamins, including cobalamin drug conjugates, naturally occurring vitamin B₁₂ and analogs of vitamin B₁₂. Specific examples of compounds suitable as a chemopotentiating agent include hydroxocobalamin, cyanocobalamin, nitrocobalamin, methylcobalamin, 5-desoxyadenosylcobalamin and nitrosylcobalamin. Radiolabelled vitamin B₁₂ compounds such as analogs, homologs and derivatives are also suitable as chemopotentiating agents. Preferably, the chemopotentiating agent is a cobalamin drug conjugate, such as nitrosylcobalamin.

Vitamin B₁₂ analogs can be synthesized in a number of ways. In addition to conjugation of the side chains of the corrin ring, conjugation to the Cbl moiety can also be made, as can conjugation to the ribose moiety, phosphate moiety, and to the benzimidazole moiety. The conjugating agent and the drug to be conjugated depend upon the type of Cbl group that is modified and the nature of the drug. One of skill in the art would understand how to adapt the conjugation method to the particular Cbl group and drug to be coupled.

Preferred methods of attaching the drug to the Cbl molecule include conjugation to Cbl via biotin. Biotin is conjugated to either the propionamide or the acetamide side chains of the corrin ring of the Cbl molecule. The initial biotin-Cbl complex can be prepared according to Pathre, et al. (Pathre, P. M., et al., “Synthesis of Cobalamin-Biotin conjugates that vary in the position in cobalamin coupling, Evaluation of cobalamin derivative binding to transcobalamin II,” incorporated by reference). Vitamin B₁₂ is commercially available in its most stable form as cyanocobalamin from Sigma Chemical (St. Louis, Mo.).

One may most easily obtain transcobalamin II in the following manner: transcobalamin II cDNA is available in the laboratories of Drs. Seetharam (Medical College of Wisconsin) and Rothenberg (VA-Hospital, New York) TC II cDNA can be expressed in a Baculovirus system to make a large amount of functionally active TC II protein (see Quadros, E. V., et al., Blood 81:1239-1245, 1993). One of skill in the art would be able to reproduce the TC II cDNA. The antibodies to TCII-R may also be obtained through the laboratory of Dr. Bellur Seetharam, Med. College of WI.

One way to make cobalamin drug conjugates is through genetic engineering. In this method, a DNA sequence encoding TC II and the peptide drug may be expressed as one chimeric molecule. For example, it is possible to generate a chimeric construct using the full-length TC II cDNA and the cDNA for a peptide drug (e.g. insulin). The chimeric construct can then be expressed to produce a fusion protein consisting of the TC II-peptide drug. Following synthesis, the chimeric protein should be tested for both TC II activity and drug activity. Cobalamin can then be allowed to bind to this chimeric protein and used for therapy.

Another embodiment of the present invention is the use of a nitric oxide donor as and chemopotentiating agent. NO donors are known in the art. NO is involved in vasodilation, and is known to contribute to increased oxidative stress, inhibition of cellular metabolism and induction of DNA damage leading to apoptosis and/or recrosis. NO donors have been found to be suitable chemopotentiating agents which sensitize cells to chemotherapeutic agents or procedures according to several embodiments of the present invention. Suitable NO donors include, but are not limited to NO-Cbl; NOC-18 (DETA NONOate, (Z)-1-[2-(2-aminoethyl)-N-(2-ammonioethyl)amino]diazen-1-ium-1,2-diolate); SNAP (S-nitroso-N-acetyl-D,L-penicillamine); and SNP (sodium nitroprusside).

DETA-NONOate, NOC-18 is a nitric oxide donor, useful for reliable generation of nitric oxide (NO) in vitro or in vivo. NOC-18 is known as (Z)-1-[2-(2-aminoethyl)-N-(2-ammonioethyl)amino]diazen-1-ium-1,2-diolate. SNAP is a suitable nitric oxide donor without any nitrate tolerance and is known as S-nitroso-N-acetyl-D,L-penicillamine. Furthermore, any additional NO donors that do not cause nitrate tolerance is a suitable chemopotentiating NO donor of the present invention, such as molsidomine and SIN-1.

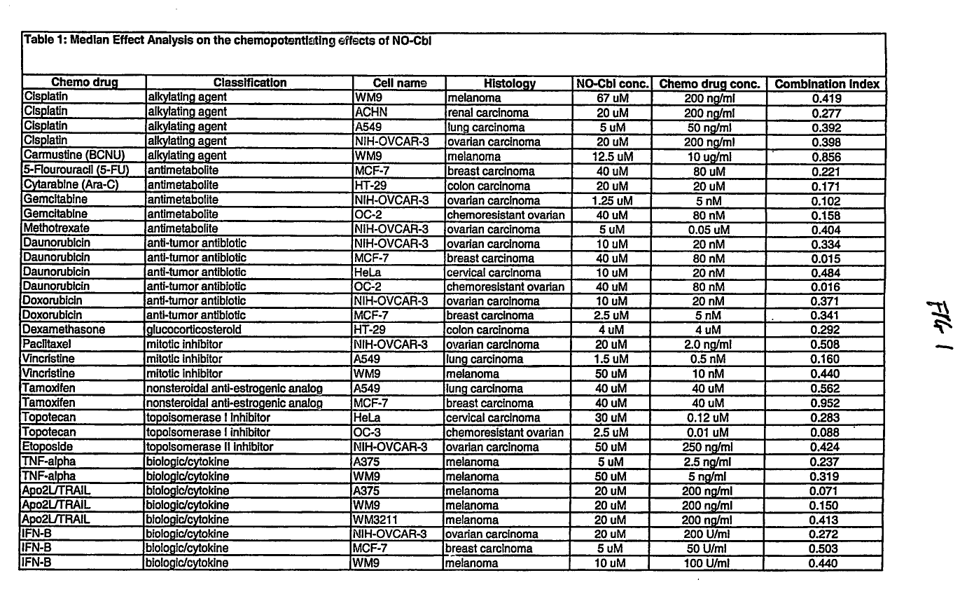

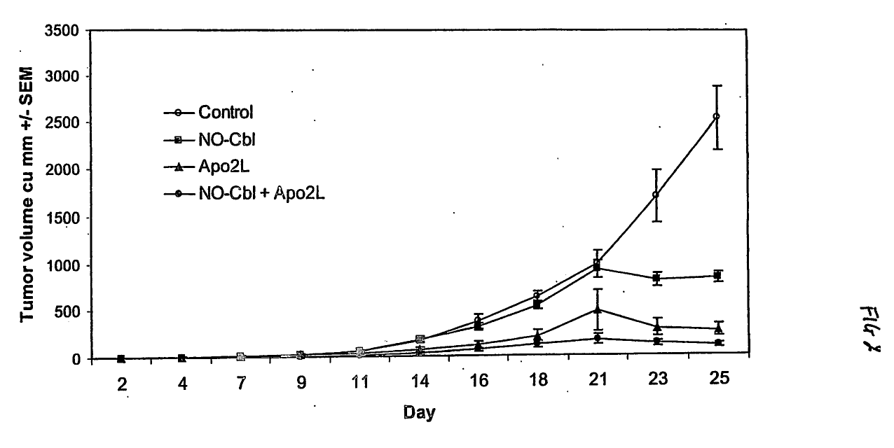

The cell survival mechanism is a vexing problem. Melanoma cells have been shown to be resistant to apoptic effects of Apo2L/TRAIL, a chemotherapeutic drug (Chawla-Sarkar. Clin. Cancer Res. (2001) and NO-Cbl (Bauer J A et al. JNCI 94(13):1010-1019 (2002) both of which are incorporate herein by reference thereto. It has been found that certain chemopotentiating agents increase the effectiveness of chemotherapeutic drugs in treating cells or conditions exacerabated by cell survival mechanisms, without adversely effecting normal cells. One such chemopotentiating agent is a cobalamin drug conjugate. Non-malignant cells were resistant to the antiproliferative effects of NO-Cbl, Apo2L/TRAIL and the combination (FIG. 1b.).

The chemopotentiating agent, chemotherapeutic agents, radiation and/or cobalamin compounds are preferably administered in effective amounts. With regard to the cobalamin or vitamin B₁₂ derived compounds, an effective amount is that amount of a preparation that alone, or together with further doses, produces the desired response. This may involve only slowing the progression of the disease temporarily, although preferably, it involves halting the progression of the disease permanently or delaying the onset of or preventing the disease or condition from occurring. This can be monitored by routine methods. Generally, doses of active compounds would be from about 0.01 mg/kg per day to 1000 mg/kg per day. It is expected that doses ranging from 50-500 mg/kg will be suitable, preferably intravenously, intramuscularly, or intradermally, and in one or several administrations per day. The administration can occur simultaneous with, subsequent to, or prior to chemotherapy or radiation so long as the chemotherapeutic agent sensitizes the system to said chemotherapy or radiation.

Such amounts will depend, of course, on the particular condition being treated, the severity of the condition and the individual patient parameters. Some parameters for consideration include age, physical condition, size and weight, the duration of the treatment, the nature of concurrent therapy (if any), the specific route of administration and like factors within the knowledge and expertise of the health practitioner. Intravenous administration and intramuscular administration avoids transport problems associated with cobalamin when administered orally. However, if the chemotherapeutic agent, such as vitamin B₁₂ analog, homolog or derivative is encapsulated, oral delivery may be preferred. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds. It is preferred generally that a maximum dose be used, that is, the highest safe dose according to sound medical judgment. Those of ordinary skill in the art will understand, however, that a patient may insist upon a lower dose or tolerable dose for medical reasons, psychological reasons or for virtually any other reason.

The chemotherapeutic agents useful according to the invention are preferably combined with a pharmaceutically-acceptable carrier that delays their release until after the tumor cells or site has been sensitized by potentiating cobalamin drug conjugates such as nitrosylcobalamin. Once sensitized, the chemopotentiating agent such as NO-Cbl may be co-administered with the chemotherapeutic agent or radiation to enhance effect. The term “pharmaceutically-acceptable carrier” as used herein means one or more compatible solid or liquid fillers, diluents or encapsulating substances which are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt. The pharmaceutical compositions also may contain, optionally, suitable preservatives, such as: benzalkonium chloride, chlorobutanol, parabens and thimerosal.

A variety of administration routes are available. The particular mode selected will depend, of course, upon the particular chemotherapeutic drug selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, intradermal, inhalation, intra-peritoneal, or parenteral routes. The term “parenteral” includes subcutaneous, intravenous, intramuscular, or infusion. Intravenous or intramuscular routes are particularly suitable for purposes of the present invention.

The pharmaceutical compositions may conveniently be presented in unit dosage form and may be prepared by any of the methods well known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for parenteral administration conveniently comprise a sterile aqueous preparation of the chemopotentiating agent (e.g. nitrosylcobalamin), which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa. which is incorporated herein in its entirety by reference thereto.

The preferred delivery systems are designed to include time-released, delayed release or sustained release delivery systems such that the delivering of the chemopotentiating or sensitizing agent occurs prior to, and with sufficient time, to cause sensitizination to the site to be treated. Thus, both the chemopotentiating agent and the chemotherapeutic agent may be delivered in a time release, delayed release, or sustained release manner such that the cell or tumor is first sensitized and then treated with an effective agent. A chemopotentiating agent may also be used in conjunction with radiation. Such systems can avoid repeated administrations of the active chemotherapeutic compound, increasing convenience to the subject and the physician, and may be particularly suitable for certain composition of the present invention.

Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-di-and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active compound is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

Use of a long-term sustained release implant may be desirable. Long-term release, are used herein, means that the implant is constructed and arranged to deliver therapeutic levels of the active ingredient for at least 30 days, and preferably 60 days. Long-term sustained release implants are well-known to those of ordinary skill in the art and include some of the release systems described above.

In one aspect of the invention, the chemotherapeutic agent is cooperatively administered with a chemopotentiating agent such as nitrosylcobalamin or a NO donor. Not all cobalamins act as chemopotentiating agents when administered alone. For example, vitamin B₁₂ itself may result in increased tumor growth. While not wishing to be bound by theory, it appears the NO cobalamin drug conjugates and NO donor in general are particularly suitable for the present invention due to the effect of NO within the cell. A major advantage of the chemopotentiating agent NO-Cbl is its tumor-specific accumulation. Cobalamin (Cbl) is avidly taken up by tumor cells relative to most normal tissues, NO-Cbl releases NO inside the cell, and therefore minimizes systemic toxicity as a result of high plasma NO concentration. Therefore suitable chemopotentiating agents as described herein do not adversely affect normal tissues, while sensitizing tumor cells to chemotherapeutic protocols. While not wishing to be bound by theory, it would appear that because the NO is released inside the cell, marked and adverse side effects such as inappropriate vasodilation or shock can be minimized. The chemotherapeutic agent is administered to the subject close enough in time with the administration of the chemopotentiating agent (e.g., a cobalamin conjugate), whereby the two compounds may exert an additive or even synergistic effect. Preferably, the composition or method is designed to allow sensitization of the cell or tumor to the chemotherapeutic or radiation therapy by administering at least a portion of the chemopotentiating agent such as a cobalamin conjugate, prior to chemotherapy and/or radiation.

A chemopotentiating agent is used in connection with a chemotherapeutic agent in several composition and method embodiments of the present invention. Suitable chemotherapeutic agents include cytokines. Cytokines are soluble polypeptides produced by a wide variety of cells. Cytokines control gene activation and cell surface molecule expression. In what follows, the term “cytokine” incorporates families of endogenous molecules of various denominations: lymphokines, monokines, interleukins, interferons, colonization factors and growth factors and peptides. The known cytokines are in particular interferon-α (IFN-α), interferon-β (IFN-β), γ-interferon (γ-IFN), interleukin-1 (IL-1) in a and β forms, interleukin-2(IL-2), interleukin-3 (IL-3), interleukin-4 (IL-4), interleukin-5 (IL-5), interleukin-6 (IL-6), interleukin-10 (IL-10), interleukin-12 (IL-12), tumor necrosis factor (TNF) in α and β forms, transforming growth factors (TGF-β), in β 1, β 2, β 3, β 1.2 forms, and colony-stimulating factors (CSF) such as the granulocyte macrophage-stimulating factor (GM-CSF), the granulocyte colony-stimulating factor (G-CSF) and the macrophage-stimulating factor (M-CSF) and the epithelial growth factor (EGF), somatostatin, endorphins, the various “releasing factors” or “inhibitory factors” such as TRF. There also exist pegilated forms of interferon. Cytokines play an essential role in the development and function of the immune system and thus in the development of an immune response.

In addition to cytokines, other chemotherapeutic agents are suitable, including but are not limited the chemotherapeutic agents described in “Modern Pharmacology with Clinical Applications”, Sixth Edition, Craig & Stitzel, Chpt. 56, pg 639-656 (2004), herein incorporated by reference. This reference describes chemotherapeutic drugs to include alkylating agents, antimetabolites, anti-tumor antibiotics, plant-derived products such as taxanes, enzymes, hormonal agents such as glucocorticoids, miscellaneous agents such as cisplatin, monoclonal antibodies, immunomodulating agents such as interferons, and cellular growth factors. Other suitable classifications for chemotherapeutic agents include mitotic inhibitors and nonsteroidal anti-estrogenic analogs. Other suitable chemotherapeutic agents include toposiomerase I and II inhibitors: CPF (8-Cyclopentyl-1,3-dimethylxanthine, topoisomerase I inhibitor) and VP16 (etoposide, topoisomerase II inhibitor).

Specific examples of suitable chemotherapeutic agents include cisplatin, carmustine (BCNU), 5-flourouracil (5-FU), cytarabine (Ara-C), gemcitabine, methotrexate, daunorubicin, doxorubicin, dexamethasone, topotecan, etoposide, paclitaxel, vincristine, tamoxifen, TNF-alpha, Apo2L/TRAIL, interferon (in both its alpha and beta forms), thalidomide, and melphalan. Other specific examples of suitable chemotherapeutic agents include nitrogen mustards such as cyclophosphamide, alkyl sulfonates, nitrosoureas, ethylenimines, triazenes, folate antagonists, purine analogs, pyrimidine analogs, anthracyclines, bleomycins, mitomycins, dactinomycins, plicamycin, vinca alkaloids, epipodophyllotoxins, taxanes, glucocorticoids, L-asparaginase, estrogens, androgens, progestins, luteinizing hormones, octreotide actetate, hydroxyurea, procarbazine, mitotane, hexamethylmelamine, carboplatin, mitoxantrone, monoclonal antibodies, levamisole, interferons, interleukins, filgrastim and sargramostim. Chemotherapeutic compositions also comprise the TNF superfamily of compounds.

Additionally, in several method embodiments of the present invention the chemopotentiating agent may be used in connection with chemo-radiation or other cancer treatment protocols used to inhibit tumor cell growth.

For example, radiation therapy (or radiotherapy) is the medical use of ionizing radiation as part of cancer treatment to control malignant cells is suitable for use in embodiments of the present invention. Although radiotherapy is often used as part of curative therapy, it is occasionally used as a palliative treatment, where cure is not possible and the aim is for symptomatic relief. Radiotherapy is commonly used for the treatment of tumors. It may be used as the primary therapy. It is also common to combine radiotherapy with surgery and/or chemotherapy. The most common tumors treated with radiotherapy are breast cancer, prostate cancer, rectal cancer, head & neck cancers, gynecological tumors, bladder cancer and lymphoma. Radiation therapy is commonly applied just to the localized area involved with the tumor. Often the radiation fields also include the draining lymph nodes. It is possible but uncommon to give radiotherapy to the whole body, or entire skin surface. Radiation therapy is usually given daily for up to 35-38 fractions (a daily dose is a fraction). These small frequent doses allow healthy cells time to grow back, repairing damage inflicted by the radiation. Three main divisions of radiotherapy are external beam radiotherapy or teletherapy, brachytherapy or sealed source radiotherapy and unsealed source radiotherapy, which are all suitable examples of treatment protocol in the present invention. The differences relate to the position of the radiation source; external is outside the body, while sealed and unsealed source radiotherapy has radioactive material delivered internally. Brachytherapy sealed sources are usually extracted later, while unsealed sources are injected into the body. Administration of the chemopotentiating agent may occur prior to, concurrently with the treatment protocol.

Apoptosis is the rigorously controlled process of programmed cell death. Current trends in cancer drug design focus on selective targeting to activate the apoptotic signaling pathways within tumors while sparing normal cells. The tumor specific properties of specific chemotherapeutic agents, such as tumor necrosis factor-related apoptosis-inducing ligand (Apo2L/TRAIL) have been reported. Apo2L/TRAIL has been used as an anti-cancer agent alone and in combination with other agents including ionizing radiation. Apo2L/TRAIL can initiate apoptosis in cells that overexpress the survival factors Bcl-2 and Bcl-XL, and may represent a treatment strategy for tumors that have acquired resistance to chemotherapeutic drugs. Apo2L/TRAIL binds its cognate receptors and activates the caspase cascade utilizing adapter molecules such as FADD. TRAIL receptors, type II membrane-bound proteins, are members of the tumor necrosis factor (TNP) superfamily of receptors. Currently, five Apo2L/TRAIL receptors have been identified. Two receptors TRAIL-R1 (DR4) and TRAIL-R2 (DR5) mediate apoptotic signaling, and three non-functional receptors, DcR1, DcR2, and osteoprotegerin (OPG) may act as decoy receptors. Agents that increase expression of DR4 and DR5 may exhibit synergistic anti-tumor activity when combined with Apo2L/TRAIL. The relative resistance of normal cells to Apo2L/TRAIL may be secondary to high expression levels of decoy Apo2L/TRAIL receptors.

The anti-tumor effects of nitrosylcobalaniin (NO-Cbl), an analogue of vitamin B₁₂ (cobalamin, Cbl) with nitric oxide (NO) as a ligand have been described. The subject of U.S. patent application Ser. No. 09/864,747, discussed compositions and methods for enhancing the uptake of cobalamin conjugates. Anti-tumor activity directly correlated with the expression of the transcobalamin II receptor (TCII-R) on the plasma membrane of cells. NO-Cbl may be used in combination with Apo2L/TRAIL because NO-Cbl induces the mRNAs of DR4, DR5, and Apo2L/TRAIL in ovarian carcinoma cells. Treatment of leukemia cells with Apo2L/TRAIL results in increased Apo2L/TRAIL mRNA and protein, suggesting autocrine regulation that can function in a positive feedback loop. Transfecting ovarian carcinoma cells with a non-functional, dominant negative DR5 receptor (DR5Δ) abrogated increases in DR4, DR5, and Apo2L/TRAIL when treated with NO-Cbl. While not wishing to be bound by theory, this suggests that the Apo2L/TRAIL receptor is necessary for the autoinduction of Apo2L/TRAIL, and that DR5Δ interferes with positive feedback signaling.

Cytokines of the TNF superfamily, upon receptor ligation, simultaneously induce an apoptotic signal (mediated via caspase-8) in addition to a survival signal (mediated by activation of nuclear factor kappa B, NF-κB). NF-κB is a transcription factor that generally functions to suppress apoptosis. Binding of TNF-α or Apo2L/TRAIL to their cognate receptors results in activation of NF-κB-inducing kinase (NIK), which phosphorylates the inhibitor of κB-kinase (IKK), resulting in the phosphorylation of IκB (inhibitor of NF-κB). Therefore, either the activation of NF-κB-inducing kinase (NIK), which further phosphorylates the inhibitor of κB-kinase (IKK) or the direct activation of the inhibitor of κB-kinase (IKK) may result in the phosphorylation of IκB (inhibitor of NF-κB). In its quiescent state, NF-κB is complexed to IκB. Upon phosphorylation IκB is degraded, allowing NF-κB to translocate to the nucleus and bind to NF-κB response elements which activate transcription. NF-κB stimulates transcription of genes such as Bcl-X_{L} and CIAP that function as survival factors. Therefore, agents that inhibit NF-κB may have anti-tumor activity.

Nitric oxide (NO) is a ubiquitous, multi-faceted signaling molecule which has been shown to inhibit NF-κB DNA binding activity and suppresses the cell survival function of NF-κB. An anti-inflammatory agent has been shown to inhibit NF-κB activity, thereby enhancing Apo2L/TRAIL-induced apoptosis in human leukemia cells. Furthermore, Apo2L/TRAIL-induced apoptosis was increased in prostate carcinoma cells that were infected with a mutant IκB, supporting the role of NF-κB as a TRAIL-induced survival factor. The use of NO-Cbl or another NO donor, to deliver nitric oxide and suppress the survival arm of NF-κB, may be used to enhance the anti-tumor effects of Apo2L/TRAIL as well as other chemotherapeutic, radiation treatment, or other anti-cancer agent as it would appear to inhibit NP-κB activity.

The chemopotentiating agent NO-Cbl exhibits tumor-specific accumulation. Cobalamin (Cbl) is avidly taken up by tumor cells relative to most normal tissues. Preferably, NO-Cbl is a NO donor suitable as a chemopotentiating agent. NO-Cbl releases NO inside the cell, and therefore minimizes systemic toxicity as a result of high plasma NO concentration. By taking advantage of the “Trojan Horse” properties of NO-Cbl, adverse side effects such as inappropriate vasodilation or shock may be minimized. NO-Cbl therefore sensitizes cancer cells to other common therapeutics.

In the following examples, cells were pre-treated with NO-Cbl to inhibit NF-κB activity and enhance the apoptotic signal of various chemotherapeutic agents. The anti-tumor effects of NO-Cbl and the chemotherapeutic agents were measured as single agents and in combination using primary and established human cancerous cell lines. The resulting experiments have shown that the chemopotentiating agents as described herein do not affect normal cells, but are effective in sensitizing cancerous cells to the various chemotherapeutic protocols. No toxicity was observed in normal cells in the resulting experiments. Unexpected synergistic effects of NO donors and cobalamin drug conjugates were observed in the following experiments.

### Materials and Methods

Synthesis of nitrosylcobalamin. Nitrosylcobalamin was synthesized as described. Hydroxocobalamin (vitamin B₁₂) acetate was dissolved in dichloromethane and exposed to CP grade NO gas at 150 psi. The reaction proceeded in a closed system within a high-pressure gas cylinder. The system was nitrogen-purged daily and evacuated prior to NO exposure. The NO gas was scrubbed prior to entering the system using a stainless steel cylinder containing NaOH pellets. The solid NO-Cbl product was collected following rotary evaporation of the solvent and stored at −80° C. prior to use.

Cell Culture treatments. Cells were maintained in RPMI or DMEM (Mediatech, Herndon, Va.) containing 5% fetal bovine serum (Hyclone, Logan, Utah) and 1% Antibiotic-Antimycotic (GEBCO, Invitrogen Carlsbad, Calif.) as recommended per the American Type Culture Collection media protocol for each cell line. Cells were maintained in 5% CO₂ at 37° C. in a humidified tissue culture incubator. Primary non-tumorigenic melanoma cell lines (DMN-1 and CMN-1), and human foreskin fibroblasts (HFF; CCF, Cleveland, Ohio) were cultured in DMEM-F12 medium supplemented with 10% FBS. Cells were confirmed as mycoplasma free.

Some experiments were performed using trimeric recombinant human Apo2L/TRAIL (Genentech Inc, San Francisco, Calif.) and were independently confirmed using recombinant Apo2L/TRAIL from another source (Peprotech Inc, New Jersey). Apo2L/TRAIL (Genentech Inc), consisted of >99% trimeric protein with Zn⁺², which is necessary for optimal biologic activity of Apo2L/TRAIL.

Other test chemotherapeutic agents tested include those listed in FIG. 1. These include cisplatin, carmustine (BCNU), 5-flourouracil (5-FU), cytarabine (Ara-C), gemcitabine, methotrexate, daunorubicin, doxorubicin, dexamethasone, etoposide, paclitaxel, vincristine, tamoxifen, topotecan, TNF-alpha, and interferon-beta.

FIG. 1 is a chart summarizing the chemopotentiating effects of NO-Cbl used in connection with a chemotherapeutic agent. The chemotherapeutic agents are listed in the column labeled “Chemo drug”. The more general classification of the chemotherapeutic agents are listed in the column labeled “classification”. The cell cultures in which the NO-Cbl and chemotherapeutic agent were introduced are listed in the column labeled “Cell name”. The concentrations of the NO-Cbl and the chemotherapeutic agent used are listed in the columns labeled “NO-Cbl conc.” and “Chemo drug conc.” respectively. The combination index illustrating the syngeristic cell proliferation affects of the NO-Cbl and chemotherapeutic drug combined are listed in the column labeled “Combination Index”. A combination index >1 indicates antagonism, =1 indicates additivity, and <1 indicates synergy.

Sulforhodamine B Cell Growth Assay. Cells were harvested with 0.5% trypsin/0.53 mM EDTA, washed with PBS and resuspended in media containing 10% FBS. Cells were plated in 96-well plates in 0.2-ml aliquots. Cells were allowed to adhere to the plate for 4 h and then NO-Cbl was added in different concentrations to the assay plate. A minimum of four were performed for each treatment. After 16 h, various chemotherapeutic agents including cytokines were added at different concentrations. Growth was monitored by the sulforhodamine B (SRB; Sigma Chemical, St. Louis, Mo.) colorimetric assay. After 36 h, the medium was removed, and the cells were fixed with 10% trichloroacetic acid and stained with SRB. Bound dye was eluted from the cells with 10 mM Tris-HCl (pH 10.5) and absorbance was measured at 570 nm using a Lab systems Multiskan RC 96-well plate reader (Lab Systems Multiscan RC, Thermo Lab Systems, Franklin, Mass.). To quantify the growth of the cells, the experimental absorbance values (Aₑₓₚ) were compared with initial absorbance readings representing the starting cell numbers (Aᵢₙᵢ). To determine the starting cell number, an additional 96-well plate was seeded with cells and fixed at the beginning of the experiment. After 5 days growth, the untreated control cells and drug treated cells were fixed and stained with SRB. The absorbances derived from the initial plate and from the untreated cells at the end of the growth period (A_{fin}) were defined as 0% and 100% growth, respectively. The percentage control growth (100%×[Aₑₓₚ−Aᵢₙᵢ]/[A_{fin}−Aᵢₙᵢ]) is expressed as a percentage of untreated controls.

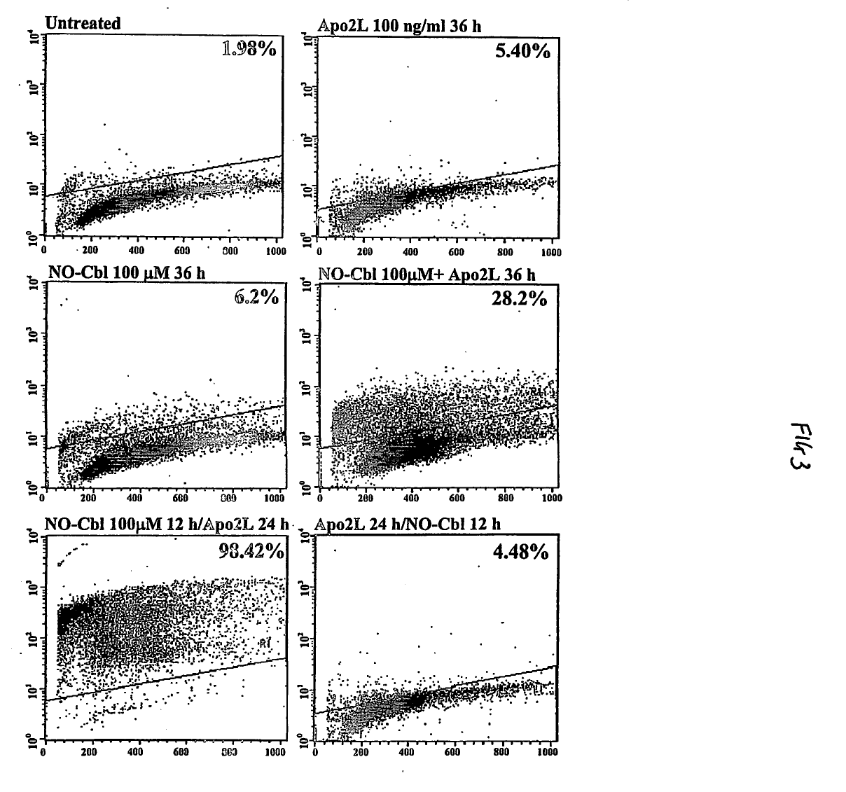

In vivo experiments. Please see FIG. 2. The Institutional Animal Care and Use Committee at the Cleveland Clinic Foundation approved all procedures for animal experimentation. Five week-old NCR male athymic nude homozygous (nu/nu) mice (Taconic, Germantown, N.Y.) were inoculated with A375 tumors. Each experimental group contained 4 mice, each mouse bearing two tumors, on opposite flanks. There were four experimental groups (untreated, single agents, and the combination). Cultured tumor cells (4×10⁶) were inoculated into flanks in the mid-axillary line. NO-Cbl was given twice daily (50 mg/kg s.c.) and recombinant trimeric Apo2L/TRAIL (50 mg/kg s.c.) was administered every other day, starting on day 2. Tumor volume was measured three times a week using the formula for a prolate spheroid: (4/3) πab² where 2a=major axis, 2b=minor axis. Formalin-fixed sections were processed by the Cleveland Clinic Histology Core. Sections were stained with hematoxylin and eosin and evaluated for pathologic changes in a blinded fashion.

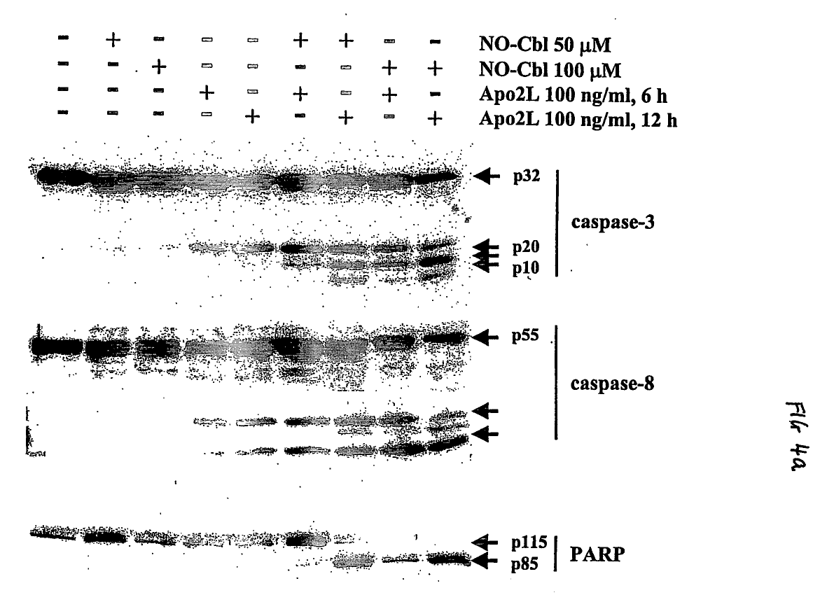

TUNEL assay. Please see FIG. 3. A375 cells were cultured for 36 h and exposed to various treatments (control, NO-Cbl, Apo2L/TRAIL and NO-Cbl+Apo2L/TRAIL. Apoptotic cells were detected by TUNEL (terminal deoxynucleotidyltransferase-mediated dUTP-biotin nick end-labeling) staining using a commercially available kit (APO-BRDU kit, BD-Pharmingen, San Diego, Calif.). Cells were processed according to the manufacturer's recommended protocol. The percentage of FITC-positive cells was analyzed by fluorescent activated cell scanning (FACS, Becton Dickinson, Facsvantage, San Diego, Calif.).

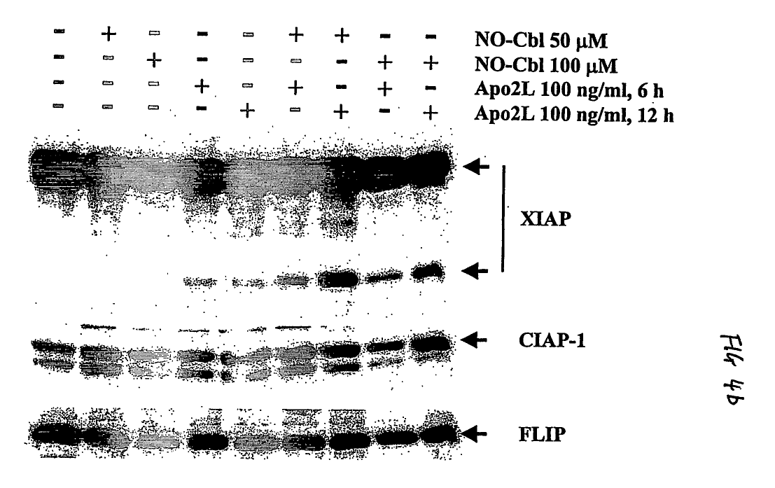

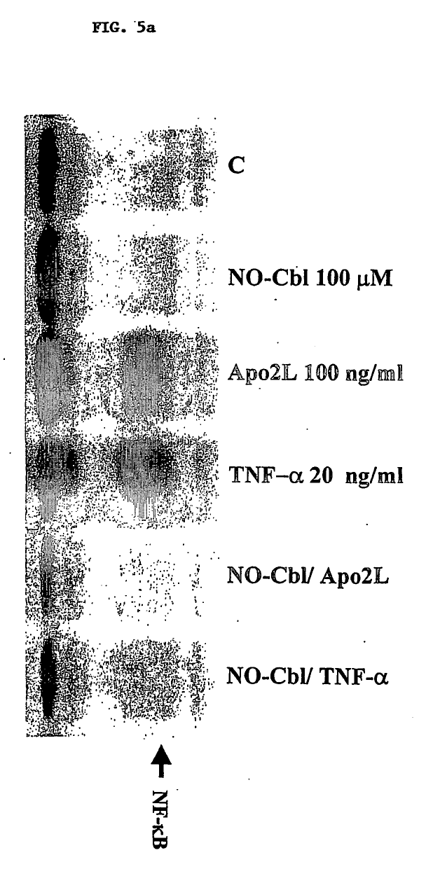

Gel Electrophoresis and Immunoblot analyses. Please see FIG. 4. Whole cell lysates were prepared in 1× lysis buffer (50 mM Tris-Cl, pH 8.0, 1% Triton×100, 10% glycerol, 1 mM EDTA, 250 mM NaCl, 1 mM DTT, 1 mM PMSF, 10 μg/ml aprotinin, 10 μg/ml leupeptin, and 10 μg/ml pepstatin) for subsequent immunoblotting studies. The SDS-PAGE was conducted by using the Laemmli buffer system and 12% polyacrylamide gels. Proteins were transferred onto PVDF membranes by the semidry method (Trans Blot SD, BioRad, Hercules, Calif.). Binding of the primary and secondary antibodies was performed according to standard protocols. Membranes were immunoblotted with pAb to caspase-3, caspase-8, XIAP (BD-Pharmingen, San Diego, Calif.), PARP (BioMOL, Plymouth Meeting, Pa.), cIAP-1, FLIP, pIκBα and IκBα (Cell Signaling, Beverly, Mass.) followed by incubation with HRP conjugated secondary antibodies (Pierce, Rockford, Ill.). Immunoreactive bands were visualized by using enhanced chemiluminescence (Perkin Elmer, Boston, Mass.). Equal protein loading was confirmed by reprobing with monoclonal anti-actin antibody (Sigma Chemicals Co, St. Louis, Mo.). All immunoblots in this study were repeated 3 times with reproducible results.

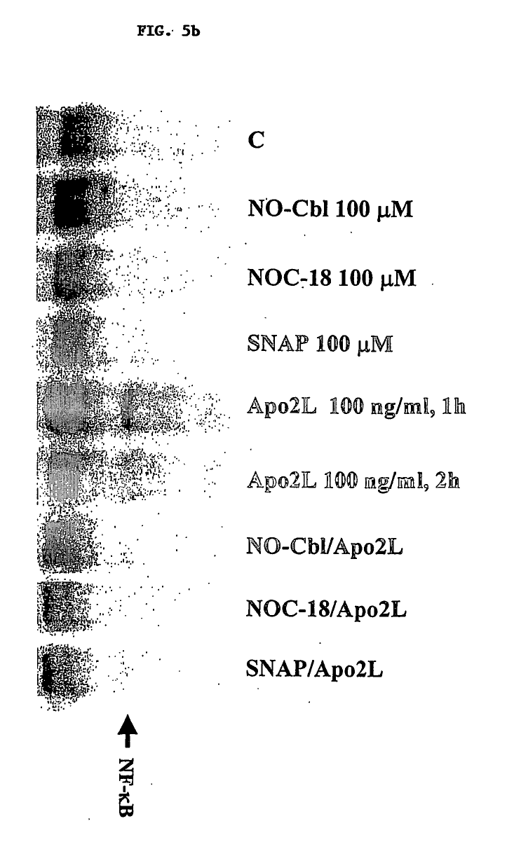

Electrophoretic mobility shift assay (EMSA). These methods apply generally to FIGS. 5, 9-10 which employ EMSA analysis. In FIG. 5, A375 cells were treated with NO donors (NO-Cbl, NOC-18, SNAP, 100 μM, 16 h), or with Apo2L/TRAIL (100 ng/ml) or TNF-α (20 ng/ml) for 1 h, or with the combination of NO donors (16 h) followed by Apo2L/TRAIL or TNF-α (1 h). Plates were washed twice with ice-cold PBS. Cells were resuspended in cold 1× lysis buffer (20 mM HEPES, 20 mM NaF, 1 mM Na₃VO₄, 1 mM EDTA, 1 mM DTT, 100 mM NaCl, 10% glycerol and protease inhibitors) and incubated on ice for 30 min followed by centrifugation at 4° C. at 10,000 rpm for 10 min. Supernatants were transferred to fresh tubes and protein concentrations were assessed using the Bradford method (Bio-RAD protein assay, BioRad, Hercules, Calif.). The NF-κB consensus binding sequence (5′AGTIGAGGGGACTTTCCCAGGC 3′) from the IFN-β gene promoter was end-labeled with γ³²P dATP (3000 Ci/mol) using T4 polynucleotide kinase. DNA binding reactions were performed in 20 μl volume containing 10 μg protein, 20 mM HEPES, 10 mM KCl, 0.1% NP-40, 0.5 mM DIT and 10% glycerol. The binding reaction was performed for 20 min at 25° C. Complexes were separated from the free probe on 6% non-denaturing polyacrylamide gels in 0.5×TBE buffer at 200 V for 2 h. Gels were dried and exposed to film.

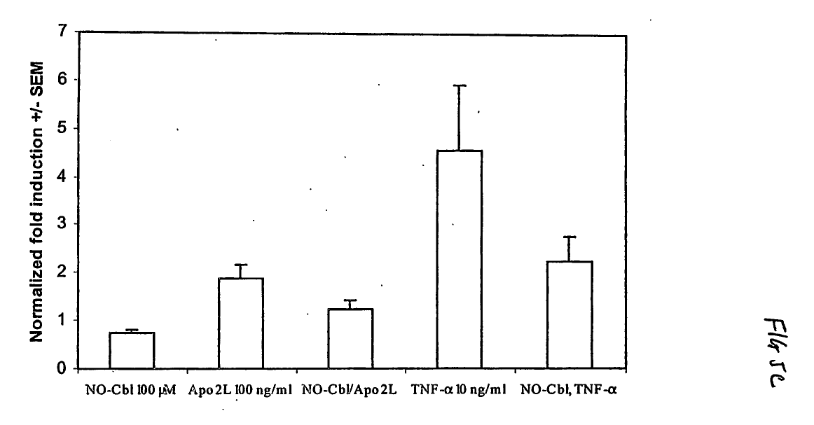

Dual Luciferase NF-κB Reporter Assay. Please see FIG. 6. The NP-κB-luciferase (NF-κB-luc) reporter plasmid, containing a 2×NF-κB response element fused to the luciferase, has been previously characterized. Renilla luciferase (pRL-TK, Promega, Madison, Wis.) vector was used to normalize for transfection efficiency. A375 cells were transfected with 20 μg NF-κB-luc and 10 μg PRL-TK using Lipofectamine plus (Gibco BRL/Life Technologies, Invitrogen Carlsbad, Calif.). After transfection cells were allowed to recover overnight and were plated in 6 well plates. Cells were pre-treated with NO-Cbl (100 μM) for 1 h followed by TNF-α (10 ng/ml) or Apo2L/TRAIL (100 ng/ml) for 4 h. Cells were then harvested in 1× passive lysis buffer and luciferase activity was measured according to the manufacturer's protocol (Promega, Madison, Wis.) using a Wallac 1420 multilabel counter (Perkin Elmer, Gaithersburg, Md.). The fold induction of NF-κB-luciferase for each treatment was based on untreated values normalized to the fold induction of pRL-TK reporter values. The assays were performed in triplicate.

### EXAMPLE 1

Anti-tumor effects of NO-Cbl, Apo2L/TRAIL, and the combination iii vitro. NO-Cbl enhances the anti-cellular effects of Apo2L/TRAIL against malignant Apo2L/TRAIL-resistant cell lines. First the antiproliferative effects of three melanoma lines A375, WM9, and WM3211 (previously reported to be resistant to Apo2L/TRAIL) were measured. Although Apo2L/TRAIL was used as the chemotherapeutic agents, a wide range of anti-cancer drugs and techniques would be enhanced by the NO based cobalamin compounds due to their effective inhibition of the cell survival mechanism. Such other chemotherapeutic agents are tested in the following examples. Three non-malignant human cell lines CMN1 and DMN1 (normal melanocytes) and fibroblasts were examined to demonstrate the tumor-specific effects of NO-Cbl and Apo2L/TRAIL. The SRB antiproliferative assay, used by the National Cancer Institute (NCI) to evaluate new chemotherapeutic agents was used herein. Median effect analysis was used to analyze drug interactions between NO-Cbl and Apo2L/TRAIL. Cells were pre-treated with NO-Cbl for 16 h followed by Apo2L/TRAIL for 24 h.

The effects of nitrosylcobalamin (NO-Cbl), Apo2L/TRAIL, and the combination on the proliferation of melanoma cell lines A375, WM9, and WM3211 and normal cell lines CMN1, DMN1, and fibroblasts were observed. Cells were treated with NO-Cbl, Apo2L/TRAIL, or pre-treated with NO-Cbl followed by Apo2L/TRAIL for three days, and growth was measured by the colorimetric sulforhodamine B assay. Data points were generated to represent the mean of four replicates±standard error of the mean (SEM). Synergy between NO-Cbl and Apo2L/TRAIL was determined by median effect analysis, (combination index >1 indicates antagonism, =1 indicates additivity, and <1 indicates synergy). The combination index is represented in FIG. 1 and as the combination index observed at the specified concentrations. The sequential treatment of NO-Cbl and Apo2L/TRAIL induced synergistic antiproliferative activity in A375, WM9 and WM3211 cells at each combined dose. Normal melanocyte cell lines CMN1 and DMN1, and normal fibroblasts were completely resistant to simultaneous NO-Cbl, Apo2L/TRAIL or the pre-treatment with NO-Cbl followed by Apo2L/TRAIL. Sequential drug treatment resulted in synergistic antiproliferative activity in all three malignant cell lines. Non-malignant cells were resistant to the antiproliferative effects of NO-Cbl, Apo2L/TRAIL and the combination. See FIG. 1.

### EXAMPLE 2

Anti-tumor effects of NO-Cbl, Apo2L/TRAIL, and the combination in vivo. To test drug activity in vivo, subcutaneous A375 xenografts were inoculated in nude mice. FIG. 2. illustrates the effect of NO-Cbl, Apo2L/TRAIL and the combination on the growth of A375 melanoma xenografts. NCR male athymic nude (nulnu) mice (n=4 per group) were injected subcutaneously with 4×10⁶ A375 cells. Drug treatments began on day two (2) after injection of tumor cells. NO-Cbl was administered twice daily for the duration of the study. Apo2L/TRAIL was administered every other day. The control mice received phosphate buffered saline. The tumor volume was measured three times per week. Data points represent the mean tumor volume (in cubic mm)±SEM. Daily drug treatments began on day 2 following implantation, at which time tumors were both visible and palpable. Untreated control tumors grew unimpeded. After 25 days, the tumors from mice treated with NO-Cbl were 67.4% smaller than the control tumors (p≦0.0002) and tumors from mice treated with Apo2L/TRAIL were 89.4% smaller than the control tumors (p≦0.00001). The tumors from mice treated with the combination of NO-Cbl and Apo2L/TRAIL were 95.7% smaller than the control tumors (p≦0.000005). Tumor regression was observed in mice treated with NO-Cbl, Apo2L/TRAIL and the combination.

Cell line A375 has a defect in endogenous TRAIL gene induction therefore, additive cellular responses from exogenous TRAIL/Apo2L were avoided. TUNEL assays of A375 cells treated in vitro with NO-Cbl, Apo2L/TRAIL, or the combination were performed. FIG. 3 illustrates a TUNEL apoptosis assay in accordance with the present invention. A375 cells were treated with NO-Cbl, Apo-2L/TRAIL, and the combination. NO-Cbl and Apo2L/TRAIL were minimally effective as single agents but demonstrated greater apoptosis when administered concomitantly. The highest levels of apoptosis were observed when cells were pre-treated with NO-Cbl followed by Apo2L/TRAIL treatment. Treatment for 36 h with NO-Cbl (100 μM) or Apo2L/TRAIL (100 ng/ml) induced 6.2% and 5.4% TUNEL-positive cells, respectively. The simultaneous co-treatment of A375 cells for 36 h with NO-Cbl (100 μM) and Apo2L/TRAIL (100 ng/ml) resulted in 28.2% TUNEL-positive cells. However, sequential pre-treatment of A375 cells with NO-Cbl (100 μM) for 12 h, followed by Apo2L/TRAIL (100 ng/ml) for an additional 24 h induced 98.4% TUNEL-positive cells, suggesting that NO-Cbl primes cells to Apo2L/TRAIL-induced apoptosis. These results are consistent with the synergistic antiproliferative effects observed in the SRB assays.

### EXAMPLE 3

Apoptosis experiments with NO-Cbl and Apo2L/TRAIL. To further examine apoptosis pathways, Western blot analysis using antibodies to various components of the apoptosis-signaling cascade was performed. A375 cells were treated with NO-Cbl (50 and 100 μM) for 16 h followed by Apo2L/TRAIL (100 ng/ml) treatment for 6-12 h. Whole cell lysates were probed for caspase-8, caspase-3, and PARP cleavage. FIG. 4 is a Western blot illustrating some of the principles of the present invention. a, A375 cells were pre-treated with NO-Cbl, followed by Apo2L/TRAIL which resulted in cleavage of caspase-3, caspase-8, and PARP. b, Sequential NO-Cbl and Apo2L/TRAIL treatment caused cleavage of XIAP, an inhibitor of apoptosis. Sequential NO-Cbl and Apo2L/TRAIL treatment caused cleavage of XIAP, an inhibitor of apoptosis. Cells pre-treated with NO-Cbl followed by Apo2L/TRAIL demonstrated enhanced cleavage of caspase-8, caspase-3 and PARP, indicating activation of initiators and effectors of apoptosis. See FIG. 4A. In addition, cleavage of the X-linked inhibitor of apoptosis (XIAP) was enhanced by NQ-Cbl pre-treatment followed by Apo2L/TRAIL, indicating that NO-Cbl promoted degradation of an apoptosis inhibitor. This effect was specific to MAP, as there was no change in levels of CIAP-1 or FLIP. See FIG. 4B.

### EXAMPLE 4

Inhibition of NF-κB survival signaling by NO-Cbl. Because NF-κB is an important cell survival regulator, we examined the effects of NO-Cbl on NF-κB DNA binding activity. The NF-κB binding sequence from the IFN-β gene promoter was used as a probe to assess DNA binding activity. A375 cells were treated with TNF-α (20 ng/ml), Apo2L/TRAIL (100 ng/ml) or NO-Cbl (100 μM). FIG. 5, illustrates an Electrophoretic Mobility Shift Assay (EMSA) of NF-κB DNA binding activity. Pre-treatment of A375 cells with NO-Cbl inhibited the NF-κB DNA binding activity induced by Apo2L/TRAIL and TNF-α. NO donors, NOC-18 and SNAP also reduced Apo2L/TRAIL induced NF-κB DNA binding. NF-κB-luc transfected A375 cells were pre-treated with NO-Cbl followed by Apo2L/TRAIL or TNF-α. Renilla luciferase was co-transfected to normalize samples for transfection efficiency. Cell lysates were analyzed for NF-κB-luc reporter activity. NO-Cbl pre-treatment inhibited Apo2L/TRAIL and TNF-α induced activation of the NF-κB luc reporter. Pre-treatment with NO-Cbl (16 h) inhibited NF-κB DNA binding activity induced by Apo2L/TRAIL and TNF-α. See FIG. 5A. Cell pre-treatment with other NO-donors including NOC-18 (100 μM) and SNAP (100 μM) also inhibited NF-κB DNA binding activity induced by Apo2L/TRAIL. See FIG. 5B. The effectiveness of these NO donors renders these compositions suitable as chemopotentiating agents.

Transient transfection assays were performed to assess NF-κB transcriptional activity. A375 cells were co-transfected with a NF-κB-luciferase reporter (NF-κB-luc) and Renilla luciferase (to assess transfection efficiency). Cells were pre-treated with NO-Cbl (100 μM) for 16 h followed by treatment with Apo2L/TRAIL (100 ng/ml) or TNF-α (10 ng/ml) for 4 hours. NO-Cbl pre-treatment caused a 34% and 51% inhibition of NF-κB activity in response to Apo2L/TRAIL and TNF-α, respectively. See FIG. 5C.

NO-Cbl treatment affected the phosphorylation state of IκBα, the prototypic inhibitor of NF-κB. Western blot analysis was performed to assess levels of phospho-IκBα and IκBα. FIG. 6 is a Western blot analysis of IκB levels and IκBα phosphorylation. IκBα and phospho-IκBα protein levels were determined in A375 whole cell lysates. Cells pre-treated with NO-Cbl exhibited decreased levels of phosphorylated IκBα following Apo2L/TRAIL or TNF-α stimulation. NO-Cbl, NOC-18, and SNAP pre-treatment all inhibited Apo2L/TRAIL-induced IκBα phosphorylation. Pre-treatment with NO-Cbl (100 μM) blocked IκBα phosphorylation induced by Apo2L/TRAIL (100 ng/ml) and TNF-α (20 ng/ml. See FIG. 6A. Total levels of IκBα were similar in all treatment groups. NOC-18 (100 μM) and SNAP (100 μM) also inhibited Apo2L/TRAIL induced phosphorylation of IκBα. See FIG. 6B.

Activation of the Apo2L/TRAIL pathway and initiation of programmed cell death. DR4 and DR5 receptors are ubiquitously expressed in malignant cells. However, Apo2L/TRAIL may be expressed at low levels in some tumor cells, which may account for differential Apo2L/TRAIL resistance. Apo2L/TRAIL resistance has also been reported in nasopharyngeal carcinomas due to a homozygous deletion of DR4. Absence of the Apo2L/TRAIL receptor may also account for resistance in a variety of melanoma cell lines. Hence, the expression ratio of Apo2L/TRAIL and its receptors may affect cellular sensitivity of malignant cell lines to Apo2L/TRAIL-induced apoptosis.

IPN-β treatment sensitized melanoma lines to the anti-tumor effects of recombinant Apo2L/TRAIL which resulted in increased expression of endogenous Apo2L/TRAIL. This endogenous production further sensitized cells to administration of exogenous Apo2L/TRAIL. Although IFN-α inhibits NF-κB activation in human leukemia cells, IFN-β did not alter the DNA binding activity of NF-κB in melanoma cells. The anti-tumor effects of IFN-β and NO-Cbl are synergistic in vitro and in vivo. Treatment with NO-Cbl increased the expression of Apo2L/TRAIL, DR4 and DR5 mRNAs, and caspase-8 enzymatic activity, indicating activation of the extrinsic apoptotic pathway.

The anti-tumor activity of NO-Cbl is also mediated by inhibition of NF-κB activation, which sensitizes cells to Apo2L/TRAIL mediated cell death. Certain renal cell carcinomas are thought to be resistant to Apo2L/TRAIL as a result of constitutively activated NF-κB. Like Apo2L/TRAIL, NO-Cbl is tumor-specific. Fibroblasts and non-tumorigenic cell lines were quite resistant to NO-Cbl (ID50's of 85-250 μM) compared to tumor cell lines (ID50's as low as 2 μM). Drug schedule is a critical determinant of the anti-tumor effects of NO-Cbl. NO-Cbl pre-treatment followed by Apo2L/TRAIL is the preferable treatment. NO-Cbl inhibits the NF-κB pro-survival arm of Apo2L/TRAIL signaling, allowing the apoptotic arm to proceed unopposed.

SNAP, SNP (nitroprusside) and NOC-18 inhibit NF-κB signaling. High concentrations of the NO donor sodium nitroprusside (SNP, 1 mM) in combination with Apo2L/TRAIL was effective at killing human colorectal carcinoma cells. The combination of SNP and Apo2L/TRAIL activated caspase-8, caspase-3 and cytochrome release which were blocked by Bcl-2, suggesting that apoptosis was mediated by the mitochondrial pathway.

### EXAMPLE 5

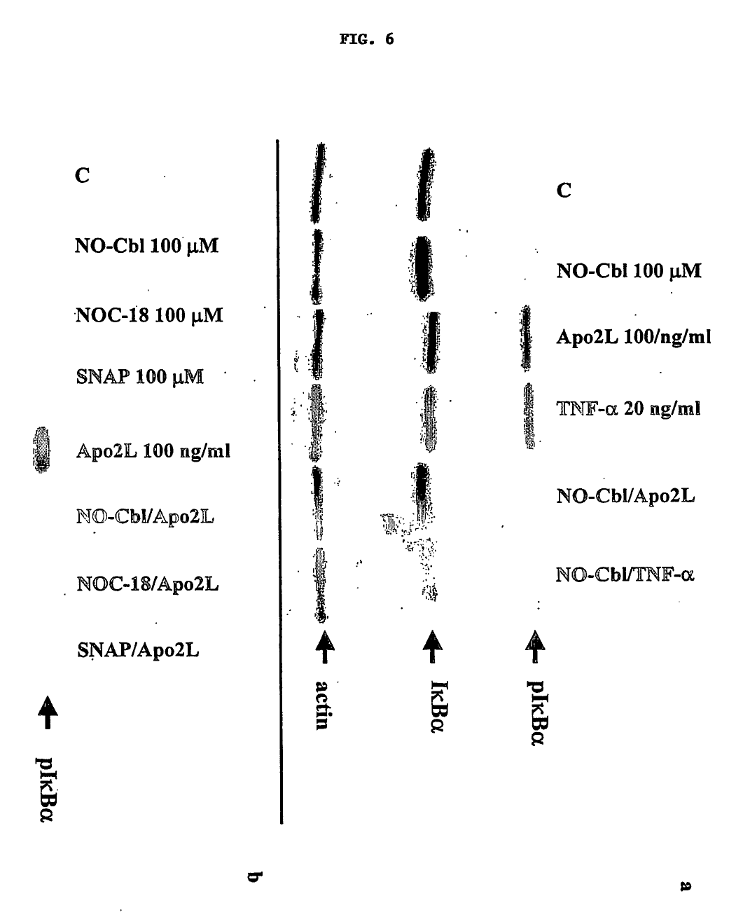

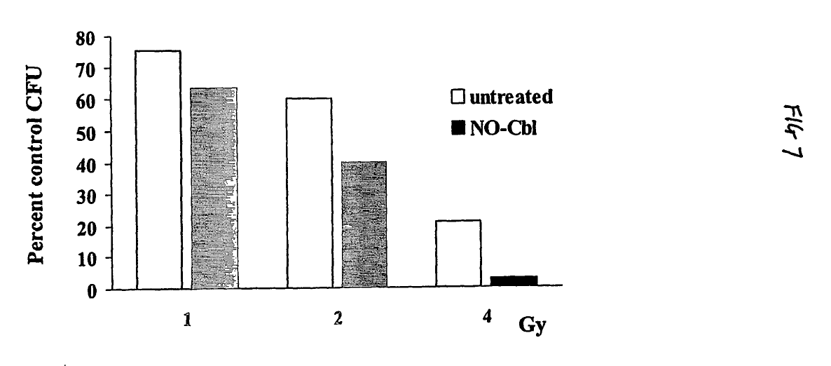

FIG. 7 illustrates that NO-Cbl sensitizes NIH-OVCAR-3 cells to gamma irradiation. NIH-OVCAR-3 cells were pre-treated with NO-Cbl (50 μM) for 16 hours. Cells were then washed and irradiated with 1, 2, and 4 Gy from a Cesium source. Cells were plated in 100 mm dishes and allowed to form colonies. Plates were examined after 23 days and colony number was determined by automated counting of stained colonies. Data is expressed as percent control colony forming units (CFU).

### EXAMPLE 6

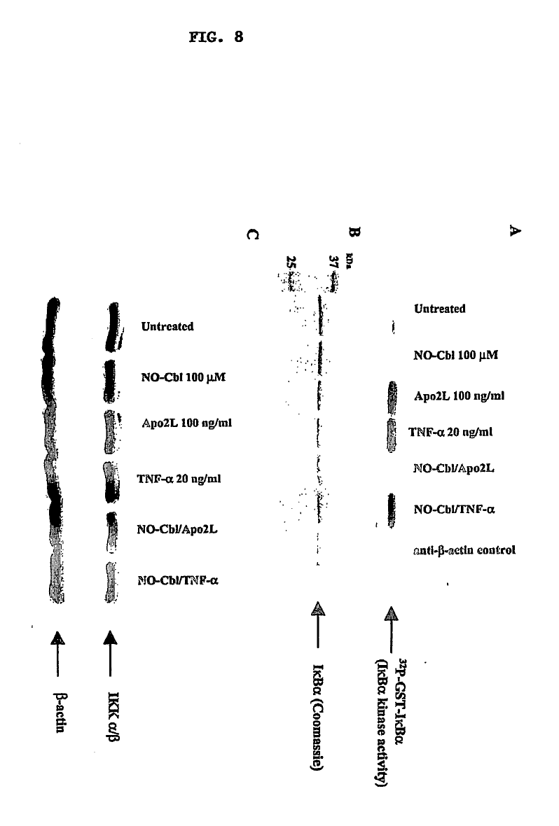

FIG. 8. illustrates how compositions according to embodiments of the present invention inhibit IKK activity. IκB kinase (IKK) activity is linked to inhibiting NF-κB, as explained. Binding of a chemotherapeutic agent to their cognate receptors results in activation of NF-κB-inducing kinase (NIK), or other “NIK-like” kinases which phosphorylates the inhibitor of κB-kinase (IKK), resulting in the phosphorylation of IκB (inhibitor of NF-κB). Therefore, either the activation of NF-κB-inducing kinase (NIK), which further phosphorylates the inhibitor of κB-kinase (IKK) or the direct activation of the inhibitor of κB-kinase (IKK may result in the phosphorylation of IκB (inhibitor of NF-κB). Agents that inhibit NF-κB have anti-tumor activity.

FIG. 8 illustrates the assessment of IκB kinase (IKK) activity using recombinant GST-IκBα-(1-54) and [γ³²P]ATP as substrates. The phosphorylated GST fusion protein was detected by autoradiography. IKK activity was determined in A375 cells pretreated with NO-Cbl followed by Apo2L/TRAIL or TNF-α stimulation for 30 minutes and 15 minutes, respectively. NO-Cbl treatment inhibited IKK activity more effectively when Apo2L/TRAIL and TNF-α were the stimulus, compared with the control. Anti-β-actin antibody served as the irrelevant antibody with no phosphorylation of GST-IκBα-(1-54) observed. Coomassie Blue-stained gel shows equal loading of GST-IκBα-(1-54) substrate. Immunoblot analysis shows the presence of equal amounts of total IKK in the lysates. β-actin was used as a loading control.

### EXAMPLE 7

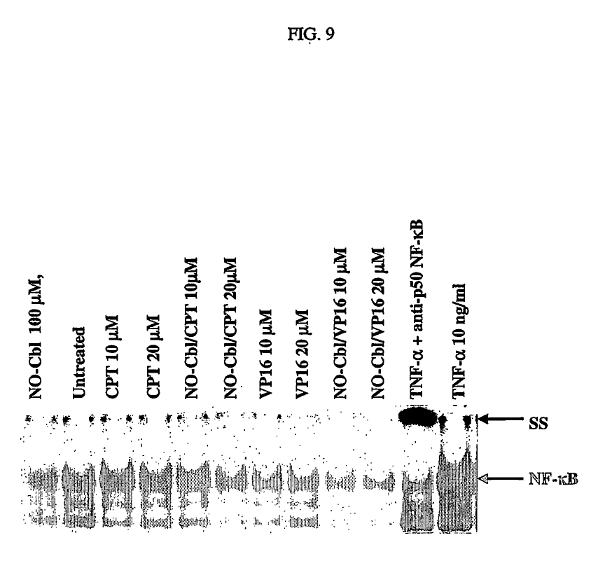

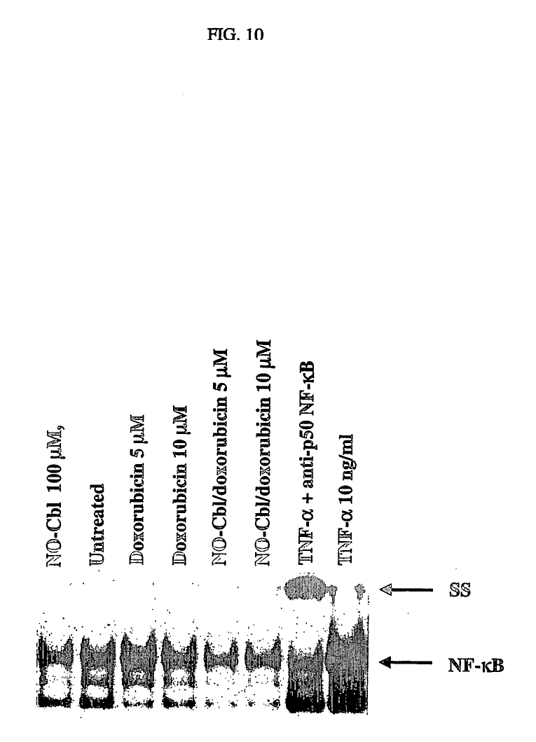

NO-Cbl inhibited NF-κB DNA binding activity as illustrated by stimulations with CPT, VP16, and doxorubicin. Electrophoretic Mobility Shift Assay (EMSA) of NF-κB DNA binding activity was conducted. Refer to FIG. 9. Pretreatment of HeLa cells (cervical carcinoma) with NO-Cbl (16 h) inhibited the NF-κB DNA binding activity induced by a two (2) hour stimulation with CPT (topoisomerase I inhibitor) or VP16 (etoposide, topoisomerase II inhibitor). Incubation with lysates with anti-NF-κB p50 antibody resulted in supershift (SS) of the NF-κB complex. TNF-α (10 min) stimulation served as a positive control of NF-κB activation. EMSA analysis has been described in reference to FIG. 5 above. NO-Cbl also sensitized cells to the effects of doxorubicin, as observed in another EMSA. Please refer to FIG. 10. As seen in FIGS. 9 and 10, NO-Cbl effectively inhibits NF-κB DNA binding activity after stimulation with CPT, VP16, and doxorubicin.

### EXAMPLE 8

Analysis of various chemotherapeutics and NO-Cbl.

According to the disclosed materials and methods as described in Example 1, similar experiments were conducted for NO-Cbl and various chemotherapeutics according to one embodiment of the present invention, in various cell lines. Single agent and combination drug effects were assessed to determine whether NO-Cbl treatment sensitized the cell line to the anti-tumor effects of the various chemotherapeutics. The cell lines were treated continuously with varying concentrations of NO-Cbl and the chemotherapeutic. Synergistic anti-proliferative activity between the various chemotherapeutics and NO-Cbl was observed across all cell lines and agents listed in FIG. 1, illustrated by the Combination Index value of less than 1. These matters are shown in the median effect analysis shown in FIG. 1 (similar to isobologram analysis) indicated synergy (a combination index <1) between NO-Cbl and the various chemotherapeutic agents.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods, and in the steps or in the sequence of steps of the method described herein, without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A therapeutic composition comprising a therapeutically effective amount of a chemopotentiating cobalamin drug conjugate.

2. The therapeutic composition of claim 1, further including a chemotherapeutic agent.

3. The therapeutic composition of claim 2, wherein the chemotherapeutic agent is selected from the group consisting of alkylating agents, antimetabolites, anti-tumor antibiotics, taxanes, hormonal agents, monoclonal antibodies, glucocorticoids, mitotic inhibitors, topoisomerase I inhibitors, topoisomerase II inhibitors, immunomodulating agents, cellular growth factors, cytokines, and nonsteroidal anti-estrogenic analogs.

4. The therapeutic composition of claim 2, wherein the chemotherapeutic agent is selected from the group consisting of cisplatin, carmustine (BCNU), 5-flourouracil (5-FU), cytarabine (Ara-C), gemcitabine, methotrexate, daunorubicin, doxorubicin, dexamethasone, topotecan, etoposide, paclitaxel, vincristine, tamoxifen, TNF-alpha, Apo2L/TRAIL, and interferon-beta.

5. The therapeutic composition of claim 1, wherein said chemopotentiating cobalamin drug conjugate is nitrosylcobalamin.

6. The therapeutic composition of claim 1, wherein said chemopotentiating cobalamin drug conjugate is selected is from the group consisting of radiolabeled vitamin B₁₂ homologs, analogs and derivatives.

7. The therapeutic composition of claim 1, wherein said chemopotentiating cobalamin drug conjugate is selected from the group consisting of hydroxocobalamin, cyanocobalamin, methylcobalamin, and 5-desoxyadenosylcobalamin.

8. The therapeutic composition of claim 1, further including a pharmaceutical carrier.

9. The therapeutic composition of claim 2, wherein the chemopotentiating agent is nitrosylcobalamin and wherein the chemotherapeutic agent is selected from the group consisting of alkylating agents, antimetabolites, anti-tumor antibiotics, taxanes, hormonal agents, monoclonal antibodies, glucocorticoids, mitotic inhibitors, topoisomerase I inhibitors, topoisomerase II inhibitors, immunomodulating agents, cellular growth factors, cytokines, and nonsteroidal anti-strogenic analogs.

10. The therapeutic composition of claim 2, wherein the chemopotentiating agent is nitrosylcobalamin and the chemotherapeutic agent is selected from the group consisting of cisplatin, carmustine (BCNU), 5-flourouracil (5-FU), cytarabine (Ara-C), gemcitabine, methotrexate, daunorubicin, doxorubicin, dexamethasone, topotecan, etoposide, paclitaxel, vincristine, tamoxifen, TNF-alpha, Apo2L/TRAIL, and interferon-beta.

11. The therapeutic composition of claim 2, wherein the chemopotentiating agent is nitrosylcobalamin and the chemotherapeutic agent is a cytokine.

12. The therapeutic composition of claim 2, wherein the chemopotentiating agent is nitrosylcobalamin and the chemotherapeutic agent is Apo2L/TRAIL.

13. The therapeutic composition of claim 2, wherein the chemopotentiating agent is nitrosylcobalamin and the chemotherapeutic agent is a member of the TNF superfamily.

14. A therapeutic composition comprising a therapeutically effective amount of a chemopotentiating nitric oxide donor and a chemotherapeutic agent.

15. The therapeutic composition of claim 14, wherein the chemopotentiating nitric oxide donor is selected from the group consisting of nitrosylcobalamin, NOC-18, SNAP and SNP.

16. The therapeutic composition of claim 14, wherein the chemotherapeutic agent is selected from the group consisting of alkylating agents, antimetabolites, anti-tumor antibiotics, taxanes, hormonal agents, monoclonal antibodies, glucocorticoids, mitotic inhibitors, topoisomerase I inhibitors, topoisomerase II inhibitors, immunomodulating agents, cellular growth factors, cytokines, and nonsteroidal anti-estrogenic analogs.

17. The therapeutic composition of claim 14, wherein the chemotherapeutic agent is selected from the group consisting of cisplatin, carmustine (BCNU), 5-flourouracil (5-FU), cytarabine (Ara-C), gemcitabine, methotrexate, daunorubicin, doxorubicin, dexamethasone, topotecan, etoposide, paclitaxel, vincristine, tamoxifen, INF-alpha, Apo2L/TRAIL, and interferon-beta.

18. A method of treating a patient with a condition in need thereof comprising sensitizing the patient to a treatment protocol by administering therapeutically effective amount of a chemopotentiating cobalamin drug conjugate and administering a treatment protocol.

19. The method of claim 18, wherein said chemopotentiating cobalamin drug conjugate is a selected from the group consisting of nitrosylcobalamin, radiolabeled vitamin B₁₂ homologs, analogs or derivatives, hydroxocobalamin, cyanocobalamin, methylcobalamin, and 5-desoxyadenosylcobalamin.

20. The method of claim 18, wherein the treatment protocol selected from the group consisting of a chemotherapeutic agent, anti-cancer agent or radiation.

21. The method of claim 20, wherein the chemotherapeutic agent is selected from the group consisting of alkylating agents, antimetabolites, anti-tumor antibiotics, taxanes, hormonal agents, monoclonal antibodies, glucocorticoids, mitotic inhibitors, topoisomerase I inhibitors, topoisomerase II inhibitors, immunomodulating agents, cellular growth factors, cytokines, and nonsteroidal anti-estrogenic analogs.

22. The method of claim 20, wherein the chemotherapeutic agent is selected from the group consisting of cisplatin, carmustine (BCNU), 5-flourouracil (5-FU), cytarabine (Ara-C), gemcitabine, methotrexate, daunorubicin, doxorubicin, dexamethasone, topotecan, etoposide, paclitasel, vincristine, tamoxifen, TNF-alpha, Apo2L/TRAIL, and interferon-beta.

23. The method of claim 18, wherein said condition is unwanted cellular proliferation.

24. A method of treating a patient with a condition in need thereof comprising sensitizing the patient to a chemotherapeutic treatment protocol by administering therapeutically effective amount of a chemopotentiating nitric oxide donor and administering a treatment protocol.

25. The method of claim 24, wherein the chemopotentiating nitric oxide donor is selected from the group consisting of nitrosylcobalamin, NOC-18, SNAP and SNP.

26. A method of inhibiting NF-κB activation comprised of administering a chemopotentiating cobalamin drug conjugate.

27. The method of claim 26, wherein said chemopotentiating cobalamin drug conjugate is a selected from the group consisting of nitrosylcobalamin, radiolabeled vitamin B₁₂ homologs, analogs or derivatives, hydroxocobalamin, cyanocobalamin, methylcobalamin, and 5-desoxyadenosylcobalamin.

28. A method of inhibiting NF-κB activation comprised of administering a chemopotentiating nitric oxide donor.

29. The method of claim 28, wherein the chemopotentiating nitric oxide donor is selected from the group consisting of nitrosylcobalamin, NOC-18, SNAP and SNP.

30. A method of treating cancer comprised of administering a composition of a chemopotentiating cobalamin drug conjugate and a chemotherapeutic agent.

31. The method of claim 30, wherein said chemopotentiating cobalamin drug conjugate is a selected from the group consisting of nitrosylcobalamin, radiolabeled vitamin B₁₂ homologs, analogs or derivatives, hydroxocobalamin, cyanocobalamin, methylcobalamin, and 5-desoxyadenosylcobalamin.

32. The method of claim 30, wherein the chemotherapeutic agent is selected from the group consisting of alkylating agents, antimetabolites, anti-tumor antibiotics, taxanes, hormonal agents, monoclonal antibodies, glucocorticoids, mitotic inhibitors, topoisomerase I inhibitors, topoisomerase II inhibitors, immunomodulating agents, cellular growth factors, cytokines, and nonsteroidal antiestrogenic analogs.

33. The method of claim 30, wherein the chemotherapeutic agent is selected from the group consisting of cisplatin, carmustine (BCNU), 5-flourouracil (5-FU), cytarabine (Ara-C), gemcitabine, methotrexate, daunorubicin, doxorubicin, dexamethasone, topotecan, etoposide, paclitaxel, vincristine, tamoxifen, TNF-alpha, Apo2L/TRAIL, and interferon-beta.

34. The method of claim 30, wherein the chemopotentiating agent is nitrosylcobalamin and wherein the chemotherapeutic agent is selected from the group consisting of alkylating agents, antimetabolites, anti-tumor antibiotics, taxanes, hormonal agents, monoclonal antibodies, glucocorticoids, mitotic inhibitors, topoisomerase I inhibitors, topoisomerase II inhibitors, immunomodulating agents, cellular growth factors, cytokines, and nonsteroidal anti-estrogenic analogs.

35. The method of claim 30, wherein the chemopotentiating agent is nitrosylcobalamin and the chemotherapeutic agent is selected from the group consisting of cisplatin, carmustine (BCNU), 5-flourouracil (5-FU), cytarabine (Ara-C), gemcitabine, methotrexate, daunorubicin, doxorubicin, dexamethasone, topotecan, etoposide, paclitaxel, vincristine, tamoxifen, TNF-alpha, Apo2L/TRAIL, and interferon-beta.

36. The method of claim 30, wherein the chemopotentiating agent is nitrosylcobalamin and the chemotherapeutic agent is a cytokine.

37. The method of claim 30, wherein the chemopotentiating agent is nitrosylcobalamin and the chemotherapeutic agent is Apo2L/TRAIL.

38. The method of claim 30, wherein the chemopotentiating agent is nitrosylcobalamin and the chemotherapeutic agent is a member of the TNF superfamily.

39. A method of treating cancer comprised of administering a composition of a chemopotentiating nitric oxide donor and a chemotherapeutic agent.

40. The method of claim 39, wherein the chemopotentiating nitric oxide donor is selected from the group consisting of nitrosylcobalamin, NOC-18, SNAP and SNP.

41. The method of claim 39, wherein the chemotherapeutic agent is selected from the group consisting of alkylating agents, antimetabolites, anti-tumor antibiotics, taxanes, hormonal agents, monoclonal antibodies, glucocorticoids, mitotic inhibitors, topoisomerase I inhibitors, topoisomerase II inhibitors, immunomodulating agents, cellular growth factors, cytokines, and nonsteroidal anti-estrogenic analogs.

42. The method of claim 39, wherein the chemotherapeutic agent is selected from the group consisting of cisplatin, carmustine (BCNU), 5-flourouracil (5-FU), cytarabine (Ara-C), gemcitabine, methotrexate, daunorubicin, doxorubicin, dexamethasone, topotecan, etoposide, paclitaxel, vincristine, tamoxifen, TNF-alpha, Apo2L/TRAIL, and interferon-beta.
